(19) United States
(12) Patent Application Publication
(10) Pub. No.: US 2023/0145905 A1
(43) Pub. Date: May 11, 2023

(54) Articulating Robotic Arm For Minimally Invasive Surgery, Surgical Robot And Method For Production

(71) Applicant: TECHNISCHE UNIVERSITÄT BERLIN, Berlin (DE)

(72) Inventors: Sven FRITSCH, Berlin (DE); Dirk OBERSCHMIDT, Oranienburg (DE)

(73) Assignee: TECHNISCHE UNIVERSITÄT BERLIN, Berlin (DE)

(21) Appl. No.: 17/981,279

(22) Filed: Nov. 4, 2022

(30) Foreign Application Priority Data
Nov. 5, 2021 (DE) 102021128809.6

(51) Int. Cl. A61B 34/30; B25J 17/00; B25J 18/00

(52) U.S. Cl. CPC A61B 34/30; B25J 17/00; B25J 18/00; A61B 2034/302

(57) ABSTRACT

An articulating robotic arm for minimally invasive surgery, comprising a proximal arm segment extending along a longitudinal axis of the robotic arm and a distal arm segment connected to the proximal arm segment, pivotable about a pivot axis orthogonal to the longitudinal axis. The distal arm segment comprises a pinion segment formed with pinion teeth arranged about the pivot axis in the form of a pinion. A rack element is arranged on the proximal arm segment such that the rack element is movable in the longitudinal direction of the rack element parallel to the longitudinal axis by means of a translation, and rack teeth formed on the rack element are engaged with the pinion teeth so that a movement of the rack element in the longitudinal direction via the rack teeth and the pinion teeth is converted into a rotational movement of the distal arm segment about the pivot axis, and so that the distal arm segment pivots relative to the proximal arm segment about the pivot axis.

The invention relates to an articulating robotic arm for minimally invasive surgery, a surgical robot, and a method for producing an articulating robotic arm.

### BACKGROUND

Robots are increasingly being used in the field of surgery. These can be so-called telemanipulators in which robotic systems directly execute a movement specified by a person performing the operation. Alternatively, robots in a narrower sense can be used, which execute movements at least partially autonomously based on prior specifications from a user and/or sensor data.

In particular, surgical robots are used in minimally invasive surgery (MIS) in which the surgical site is accessed through small incisions and/or natural body openings. In this case, surgical robots can be used to at least partially compensate for the limited mobility of a person performing the surgery due to constricted access. This is particularly important in so-called single-port procedures, in which access to the surgical site is through a single access point. In single-port laparoscopy (SPL), for example, a single incision is made through the navel to introduce all the instruments required for the operation into the abdominal cavity.

In the case of robotic arms for surgical robots, a distinction can be made between discrete and flexible robotic structures. While discrete robots are based on joints connected in series by rigid connections, continuous curvatures are possible with flexible robots, for example by means of a curved wire.

In the case of serial sequences of joints and rigid connections in the sense of a discrete robot structure, it is known, for example, to achieve angular adjustment by means of wires which are used to transmit pulling forces and, if necessary, pushing forces to make the joints rotate, cf., for example, Thant, Z. M. et al: Ergonomic Master Controller for Flexible Endoscopic Gastrointestinal Robot Manipulator, Intl. Conf. on Biomedical and Pharmaceutical Engineering, 2006. Alternatively, angle adjustment can be done via embedded electric motors that accomplish tilt via a gear-and-belt combination (see Vitiello, Valentina et al.: Emerging Robotic Platforms for Minimally Invasive Surgery, IEEE Reviews in Biomedical Engineering, 2013, vol. 6), or by means of a mechanism of shafts moving inside each other (see Piccigallo, Marco et al.: Design of a Novel Bimanual Robotic System for Single-Port Laparoscopy, IEEE/ASME Transactions on Mechatronics, 2010, vol. 15, 6). An angular adjustment mechanism according to Lehman, Amy C. et al: Natural orifice cholecystectomy using a miniature robot, Surgical Endoscopy, 2009, is composed of two so-called linkages that rotate two side elements via an electric motor embedded in a center element.

In flexible robots, actuation with NiTi wires has become widely accepted. For example, such wires can be used to open flaps on robotic arms that deflect instruments or instrument arms (cf. Bardou, Berengere et al.: Design of a telemanipulated system for transluminal surgery, 31st Annual International Conference of the IEEE EMBS, 2009; Swanstrom, Lee et al.: Development of a New Access Device for Transgastric Surgery, The Society for Surgery of the Alimentary Tract, 2005; and De Donno, Antonio et al: Introducing STRAS: a New Flexible Robotic System for Minimally Invasive Surgery, 2013 IEEE International Conference on Robotics and Automation (ICRA), 2013). Angular adjustment can also be achieved using a coiled cable, which, unlike a NiTi wire, can only withstand tension rather than tension and compression. Such a cable can be wound around a post with one turn. On an opposite side, the cable can be wrapped around a second post in the opposite direction, so that when the cable is pulled on one side while the cable is allowed to trail on the other side, a robot arm will tilt in the corresponding direction. This allows rotation in both directions (cf., for example, Can, Salman et al: Design, Development and Evaluation of a Highly Versatile Robot Platform for Minimally Invasive Single-Port Surgery, The Fourth IEEE RAS/EMBS International Conference on Biomedical Robotics and Biomechatronics, 2012.).

In a concept with a plurality of segments (for example, according to Ouyang, Bo et al.: Design of a three-segment continuum robot for minimally invasive surgery, Robotics and Biomemetics, 2016), angular adjustment can be achieved by bending the first segment, i.e., the segment closest to a base plate, while bending the second segment in the opposite direction. There are similar concepts, most of which have only two serial segments (cf. Xu, Kai et al.: Development of the SJTU Unfoldable Robotic System (SURS) for Single Port Laparoscopy, IEEE/ASME Transactions on Mechatronics 1, 2015). In particular, it can be possible to form an S-curve. Angular adjustment can in particular be achieved by targeted curving of individual segments that are made of NiTi wire. In addition, it can be provided to push apart several robotic arms of a robot, for example by means of a parallelogram mechanism. In an exemplary parallelogram mechanism (see Xu, Kai et al.: System Design of an Insertable Robotic Effector Platform for Single Port Access (SPA) Surgery, The 2009 IEEE/RSJ International Conference on Intelligent Robots and Systems, 2009), pulling back a rail causes the parallelogram arm to expand, while moving the rail forward causes the flexible robot part to lay flat.

Another possibility to adjust angles in flexible robots is to use chambers that can be filled with a fluid (e.g., compressed air or water). Such a concept is known, for example, from Chen, G. et al.: Development and kinematic analysis of a silicone rubber bending tip for colonoscopy, Proceedings of the 2006 IEEE/RSJ International Conference on Intelligent Robots and Systems, 2006.

In robotic systems, a conversion from linear to rotational movement or vice versa is often required. Herein, it is known, to generate a linear movement from a rotational movement, to use rack-and-pinion pairs in which the pinion, which is mounted on a motor shaft, drives the rack to achieve a linear movement. In doing so, high smoothness, positioning accuracy and feed force can be achieved. In machines (for example, construction machines, machine tools, bending machines, mold carriers, foundry, mining, agricultural and packaging machines) that provide linear movement with high force, in particular by means of oil pressure, piston rotary drives are known in which oil pressure causes the displacement of racks, thereby converting the linear movement of the racks into a rotational movement of the pinion via gearing.

From document WO 03/013349 A1, it is known to angularly deflect a tip of an image probe by means of a rack-and-pinion pair. The document DE 10 2015 114 742 B4 describes a movement of forceps jaws of an endoscopic instrument via a rack and pinion.

The document DE 10 2010 034 380 A1 discloses a joint section of an endoscope shaft for an articulating connection of a distal section of an endoscope shaft to a proximal section of the endoscope shaft. The distal shaft section is connected to the proximal shaft section via two rigid joint rods which connect to the distal and the proximal shaft section with a respective joint. The article “Besserer Durchblick für den Chirurgen” (Better overview for the surgeon, available under https://robotik-produktion.de/allgemein/besserer-durchblick-fuer-den-chirurgen/) describes a robotic arm for holding and moving an endoscope during surgery.

### SUMMARY

It is an object of the invention to specify improved technologies for surgical robots with articulating robotic arms, wherein in particular mechanical complexity is low and an efficient operation is ensured.

The object is achieved by an articulating robotic arm according to independent claim 1 as well as a surgical robot and a method for producing an articulating robotic arm according to further independent claims. Embodiments are subject matter of dependent claims.

According to one aspect, an articulating robotic arm for minimally invasive surgery is provided. The articulating robotic arm comprises a proximal arm segment extending along a longitudinal axis of the robotic arm, and a distal arm segment connected to the proximal arm segment so to be pivotable about a pivot axis which pivot axis is orthogonal to the longitudinal axis. The distal arm segment has a pinion segment formed with pinion teeth arranged about the pivot axis in the form of a pinion. A rack element is arranged on the proximal arm segment such that the rack element is movable in the longitudinal direction of the rack element parallel to the longitudinal axis of the robotic arm by means of a translation, and rack teeth formed on the rack element are engaged with the pinion teeth such that movement of the rack element in the longitudinal direction via the rack teeth and the pinion teeth is converted into rotational movement of the distal arm segment about the pivot axis, and the distal arm segment pivots relative to the proximal arm segment about the pivot axis.

According to another aspect, a surgical robot is provided, in particular for minimally invasive surgery, comprising an articulating robotic arm according to the disclosure.

According to yet another aspect, a method for producing an articulating robotic arm is provided. The method comprises the steps of providing a proximal arm segment, the direction of extent of which defines a longitudinal axis of the robotic arm, providing a distal arm segment having a pinion segment formed with pinion teeth arranged about a pivot axis in the form of a pinion, connecting the distal arm segment to the proximal arm segment such that the distal arm segment is pivotable about the pivot axis relative to the proximal arm segment and the pivot axis extends orthogonal to the longitudinal axis, and arranging a rack element on the proximal arm segment. The rack element is arranged on the proximal arm segment such that the rack element is movable in the longitudinal direction of the rack element parallel to the longitudinal axis by means of translation, and rack teeth formed on the rack element are engaged with pinion teeth such that a movement of the rack element in the longitudinal direction via the rack teeth and the pinion teeth is converted into a rotational movement of the distal arm segment about the pivot axis and the distal arm segment pivots relative to the proximal arm segment about the pivot axis.

In the context of the present disclosure, the terms robot and robotic arm refer both to robots in the narrower sense, which perform autonomous or semi-autonomous movements, and to telemanipulation systems in which robotic arms perform a movement specified directly by an operator. Terminologically, combinations of the two concepts are also covered.

In the context of the articulating robotic arm and the surgical robot, the term proximal denotes a direction or arrangement facing a central main part of the robot. The term distal denotes a direction or arrangement facing away from the central main part and facing towards the surgical site.

Surprisingly, it has been found that a rack-and-pinion pair according to the independent claims, which is formed by the rack element and the pinion segment, can provide sufficient stability and force for use in an articulating robotic arm for minimally invasive surgery, while at the same time, suitable component dimensioning for this application is provided. In contrast, it is in accordance with the prior art, for example according to the above-mentioned documents WO 03/013349 A1 and DE 10 2015 114 742 B4, to use sufficiently miniaturized rack-and-pinion pairs for use in robotic arms for minimally invasive surgery exclusively for applications with low force requirements. In comparison, an angular deflection of a robotic arm, which must also function reliably when the robotic arm is subjected to the loads that can be expected in use, requires a high level of force input. According to the disclosure, an angular deflection of an articulating robotic arm can be provided without using cables or wires for this angular deflection. Herein, cables or wires can be provided for additional movements and/or degrees of freedom of the robot arm.

The pinion teeth can be formed as formations of protrusions of the distal arm segment. In this case, the pinion segment forms a section of the distal arm segment. For example, the pinion teeth can be produced together with the distal arm segment by means of an additive method, in particular 3D printing, the pinion teeth can be molded onto the distal arm segment, or the pinion teeth can be formed out of the distal arm segment, for example by means of machining such as milling or by punching or cutting, for example laser or water jet cutting. By forming the pinion teeth as formations (protrusions) of the distal arm segment, the number of fastening elements required and the associated material costs and assembly time can be reduced.

Alternatively, the pinion teeth can be produced separately and attached to the distal arm segment, for example by means of adhesive bonding, welding, soldering, screws or rivets. In this case, the pinion segment forms a separate element arranged on and attached to the distal arm segment. In particular, a pinion or a section of a pinion (in particular in the form of a circular segment with teeth along the circumference) can be arranged on the distal arm segment and firmly connected thereto. In this manner, the use of standard components is made possible, thereby reducing production costs.

The pinion teeth can extend about the pivot axis over a limited angular range. In particular, as a result of this, a section of a pinion can be formed and/or arranged on the distal arm segment with the pinion segment. The limited angular range can correspond to a radius of movement of the angular deflection of the robotic arm. For example, the pinion teeth can span an angle between a full straightening of the distal arm segment relative to the proximal arm segment (corresponding to 0°) and a right angle between the proximal arm segment and the distal arm segment (90°).

The distal arm segment can include a second pinion segment with pinion teeth arranged about the pivot axis, which is arranged along the pivot axis spaced apart from the pinion segment on the distal arm segment. In particular, the pinion segments can be arranged on opposite sides of the distal arm segment along a direction determined by the pivot axis. As a result, rack teeth engage with both pinion segments to pivot the distal arm segment. By providing a second pinion segment, in particular on opposite sides of the distal arm segment, jamming and/or tilting of the angular deflection mechanism can be prevented or reduced. Further, with an embodiment having a second pinion segment, a safety mechanism can be provided to allow safe engaging and removal of the robot by the remaining, still intact pinion segment after a possible tooth breakage of one of the pinion segments.

The rack element can have two sets of rack teeth, the two sets of rack teeth being parallel and spaced apart in the direction of the pivot axis such that the first set of rack teeth engages the pinion teeth of the pinion segment and the second set of rack teeth engages the pinion teeth of the second pinion segment.

Alternatively, for embodiments having a second pinion segment, it can be provided that the rack element includes only one set of rack teeth, the rack teeth being configured to be wide enough to engage both pinion segments in order to pivot the distal arm segment.

The rack element can be formed with a proximal rack segment and a distal rack segment connecting to the proximal rack segment in the longitudinal direction, the distal rack segment being wider in the direction of the pivot axis than the proximal rack segment. Here, the rack teeth can be arranged in the region of the distal rack segment. The proximal rack segment can be free of rack teeth. In embodiments having two sets of rack teeth, the sets of rack teeth can be arranged parallel to and spaced apart from each other in the direction of the pivot axis on the distal rack segment.

The proximal arm segment can have a guide channel extending along the longitudinal axis in which the rack element, in particular with a proximal rack segment, is arranged and guided at least in sections. For example, the guide channel can have a V-shape or U-shape in cross-section. The guide channel can extend over the entire length of the proximal arm segment. Alternatively, the guide channel may extend only partially along the proximal arm segment, with the length of the guide channel corresponding to at least a length by which the rack element is movable parallel to the longitudinal axis for maximum angular deflection of the distal arm segment. In this case, the rack element can comprise a protruding guide element arranged in the guide channel and linearly guided therein.

Additionally or alternatively to a guide channel of the proximal arm segment, the proximal arm segment can comprise an arm segment guide element which is preferably arranged at a distal end of the proximal arm segment and projects in the longitudinal direction, in particular integrally molded on the proximal arm segment or formed as a separate element and attached to the proximal arm segment. The arm segment guide element can be formed as a guide rod. In an embodiment of the articulating robotic arm with arm segment guide element, the rack element has a rack element guide channel in which the arm segment guide element is arranged and guided at least in sections. The rack element guide channel can be arranged on a side of the rack element opposite the rack teeth, in particular on a side of the distal rack element opposite the rack teeth. The arm segment guide element and the rack element guide channel preferably have a non-circular cross-section, in particular a rectangular cross-section.

By guiding the relative movement between the proximal arm segment and the rack element by means of the guide channel and/or the rack element guide channel, safe guidance and smooth running can be ensured, in particular due to the fact that side walls of the guide channel and/or the rack element guide channel engage around the rack element and/or the arm segment guide element. By means of a guide in the region of or at a short distance from the rack teeth, acting radial forces and/or tangential forces of the rack-and-pinion pair of the articulating robotic arm can be absorbed in the elements of the guide, whereby in particular a bending moment acting on the rack element can be reduced. Thus, a mechanical load on the rack element can be reduced.

The distal arm segment can be formed, at least in sections, as a flexible arm segment that can be angularly deflected continuously or quasi-continuously along its longitudinal extent. Here, the distal arm segment distal to a region in which the pinion segment is arranged can be formed according to a flexible robot structure, for example according to the principles of flexible robot structures known as such from the prior art, or according to flexible robot structures deviating from the prior art.

In a preferred embodiment, the flexible arm segment can be formed with at least three pull-push elements or pull-push means that are arranged parallel to each other along a longitudinal extent of the flexible arm segment and movable along the longitudinal extent. Each pull-push element/means can be assigned a corresponding linear actuator which is configured to displace the respective pull-push means along the longitudinal extent of the flexible arm segment. Here, the respective linear actuators can be part of the articulating robotic arm, or the respective push-pull means can be configured to be functionally connected to a corresponding linear actuator arranged outside the articulating robotic arm, for example as part of a surgical robot to which the articulating robotic arm is assigned. A linear actuator can be an actuator that directly provides a linear movement or an actuator that provides another form of movement, for example a rotation, in conjunction with a mechanism that converts the movement provided by the actuator into a linear movement.

The push-pull elements can be wires, in particular NiTi wires, i.e., wires made of a nickel-titanium alloy. The wires can have a sufficiently high buckling stiffness to be used as pull-push elements or means in both a pull and a push mode. When the flexible arm segment is angularly deflected or bent, the length of a pull-push element located centrally with respect to the cross-section of the flexible arm segment can remain constant, thus the corresponding linear actuator may not move, while one or more of the remaining pull-push elements are moved, thus are displaced along the longitudinal extent of the flexible arm segment.

According to a preferred embodiment, the flexible arm segment can be formed with at least five push-pull means or elements, wherein the configurations explained above can be provided accordingly with respect to the push-pull elements. In particular, when the flexible arm segment is angularly deflected or bent, the length of a push-pull element arranged centrally with respect to the cross-section of the flexible arm segment can remain constant while one or more of the remaining push-pull elements are moved, thus are displaced along the longitudinal extent of the flexible arm segment. In this case, two of the non-central push-pull elements can be antagonistically actuated so that one push-pull element is pushed in a distal direction while the other push-pull element is pulled in a proximal direction on an opposite side with respect to the central push-pull element so that a bending of the flexible arm segment is achieved.

In an embodiment of the distal arm segment configured at least in sections as a flexible arm segment, the distal arm segment can be formed with a fixed segment which is connected to the proximal arm segment to be pivotable about the pivot axis and which comprises the pinion segment, the flexible arm segment being directly or indirectly connected to the fixed segment. Here, in particular, pull-push elements of the flexible arm segment can be guided by the proximal arm segment and the fixed segment of the distal arm segment. The fixed segment can have element guide channels for guiding the pull-push elements. By means of the element guide channels, a distribution of the pull-push elements in the distal arm segment can be achieved that is different from that in the proximal arm segment. For example, it can be provided that the pull-push elements in the proximal arm segment are guided to be arranged directly linearly next to each other while they are arranged along the distal arm segment corresponding to a flexible arm segment, in particular circularly around a central pull-push element. For this purpose, it can be provided that an arrangement of the element guide channels corresponds to an arrangement of the pull-push elements in the distal arm segment, so that the pull-push elements, before entering the element guide channels, are rearranged with respect to their arrangement in the proximal arm segment. Alternatively, the element guide channels can be configured to directly guide a rearrangement of the pull-push elements. In this case, the arrangement of inlets of the element guide channels facing the proximal arm segment corresponds to an arrangement of the pull-push elements in the proximal arm segment, and an arrangement of outlets of the element guide channels facing away from the proximal arm segment corresponds to an arrangement of the pull-push elements in the distal arm segment.

Alternatively or in addition to a flexible arm segment, the distal arm segment can be formed, at least in sections, as a discrete-robotic arm segment having segments arranged one behind the other along its longitudinal extent, wherein the discrete-robotic arm segment can be angularly deflected in segments along its longitudinal extent. In this case, the discrete-robotic arm segment distal of a region in which the pinion segment is arranged can be formed in accordance with a discrete robot structure, for example in accordance with the principles of discrete robot structures known as such in the prior art, or in accordance with discrete robotic structures different from the prior art. In embodiments in which the distal arm segment comprises both at least one flexible arm segment and at least one discrete robotic arm segment, the flexible arm segment and the discrete-robotic arm segment can be arranged one behind the other in a suitable order along the longitudinal extent of the distal arm segment, wherein both the flexible arm segment and the discrete-robotic arm segment are formed distal of a region in which the pinion segment is arranged, in particular distal of a fixed segment of the distal arm segment.

The articulating robotic arm can include a linear actuator connected to the rack element and configured to translationally move the rack element back and forth parallel to the longitudinal axis. Alternatively, the rack element can be configured to be functionally connected to a corresponding linear actuator that is arranged outside the articulating robotic arm, for example as part of a surgical robot to which the articulating robotic arm is assigned. A linear actuator can be an actuator that directly provides a linear movement or an actuator that provides another form of movement, for example a rotation, in connection with a mechanism that converts the movement provided by the actuator into a linear movement.

The distal arm segment can be connected to the proximal arm segment by means of a screw connection so that it can pivot about the pivot axis. Alternatively or additionally, another type of connection, for example a riveted connection, a connection via a separate joint component or a direct and rotationally movable positive fit connection between the distal arm segment and the proximal arm segment can be provided. The proximal arm segment can be formed with a connection protrusion that extends to and is pivotally connected to the distal arm segment. In particular, it can be provided that the connection protrusion extends between two pinion segments of the distal arm segment and is pivotally fixed therebetween. Here, the pinion segments can be formed as protrusions of flank elements of the distal arm segment, and the connection protrusion can extend between the flank elements and be pivotally fixed therebetween. The connection protrusion can be formed integrally with the proximal arm segment or formed separately and fixedly attached thereto.

The surgical robot can be formed as a robot that may be assembled inside the body of a patient. In this regard, it can be provided that individual elements of the surgical robot, for example comprising a plurality of robotic arms, are separately inserted into the patient's body and connected to one another in the patient's body to form the surgical robot. In one configuration of the surgical robot as a robot for assembly in a patient's body, it is made possible to insert the surgical robot into the patient's body through a smaller incision in the patient's abdominal wall than would otherwise be possible because the surgical robot is inserted into the patient's body in a plurality of separate elements. For example, it can be provided to insert multiple robotic arms, preferably four robotic arms, into the patient's body and then to arrange a housing in the access to the patient's body. Subsequently, the robotic arms can be arranged and fixed in the housing to mount the mountable robotic arm. In this case, a sealing fixing element can be provided, which, after arranging the robotic arms in the housing, is introduced into the housing from outside the patient's body and encloses the robotic arms in a sealing and fixing manner.

In connection with the method for producing an articulating robotic arm, providing the distal arm segment can comprise stamping a sheet metal and folding the stamped sheet metal into the distal arm segment. Alternatively, providing the distal arm segment can comprise producing the distal arm segment, at least in sections, by means of an additive manufacturing process. For example, it can be provided to produce the distal arm segment by means of a 3D printing process. In particular, the pinion segment can be 3D printed. Alternatively or additionally, any or all of the elements of the articulating robotic arm can be produced by means of an additive manufacturing process, such as 3D printing. Accordingly, it can be provided for the articulating robotic arm that one, some, or all of the elements are produced by means of an additive manufacturing process, such as 3D printing.

Accordingly, the disclosure includes a computer program product comprising commands which, when the program is executed on an additive manufacturing device, in particular a 3D printer, cause additive manufacturing of the articulating robotic arm or components thereof.

The exemplary embodiments explained above in connection with the articulating robotic arm can be provided accordingly for the surgical robot as well as the method for producing an articulating robotic arm, and vice versa.

### DESCRIPTION OF EXEMPLARY EMBODIMENTS

In the following, further exemplary embodiments are explained in more detail with reference to figures of a drawing. In the figures:

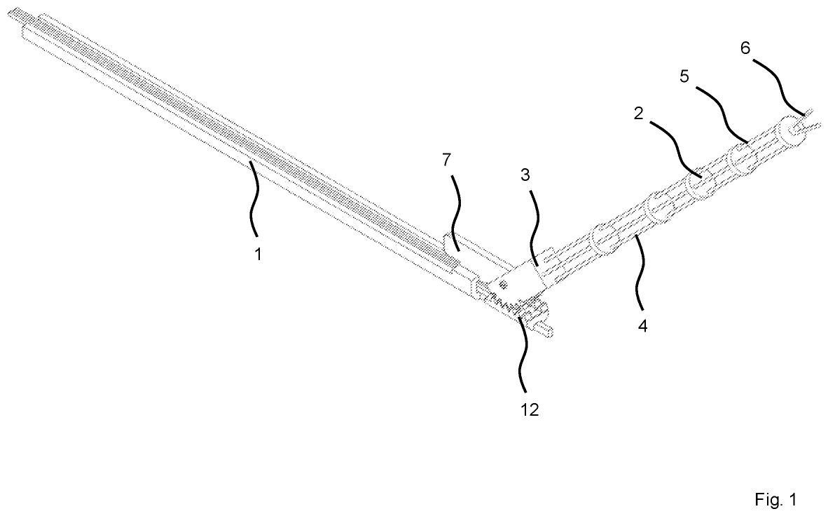

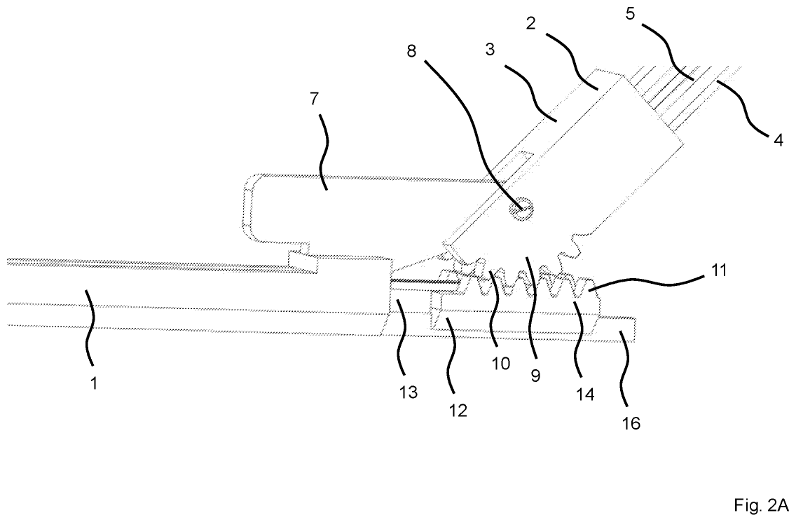

FIG. 1 shows a schematic illustration of an articulating robotic arm for minimally invasive surgery;

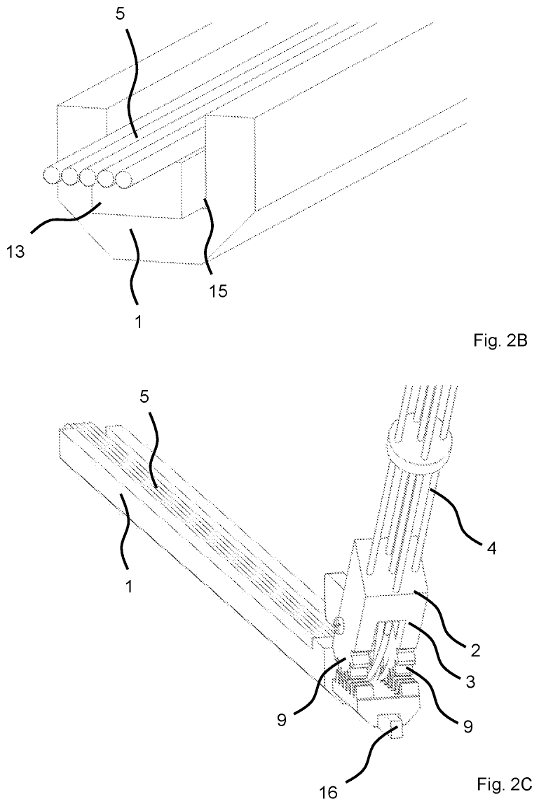

FIG. 2A shows a schematic detail view of the articulating robotic arm according to FIG. 1;

FIG. 2B shows another schematic detail view of the articulating robotic arm according to FIG. 1;

FIG. 2C shows yet another schematic detail view of the articulating robotic arm according to FIG. 1;

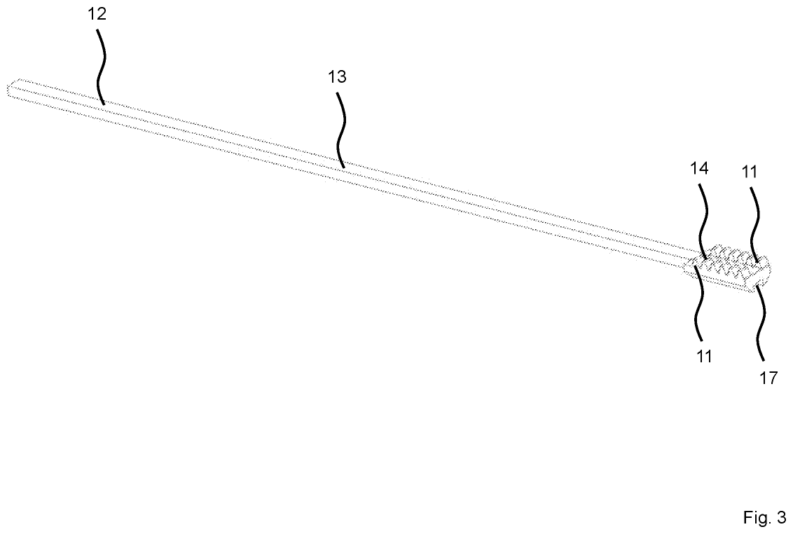

FIG. 3 shows a schematic illustration of a rack element of an articulating robotic arm;

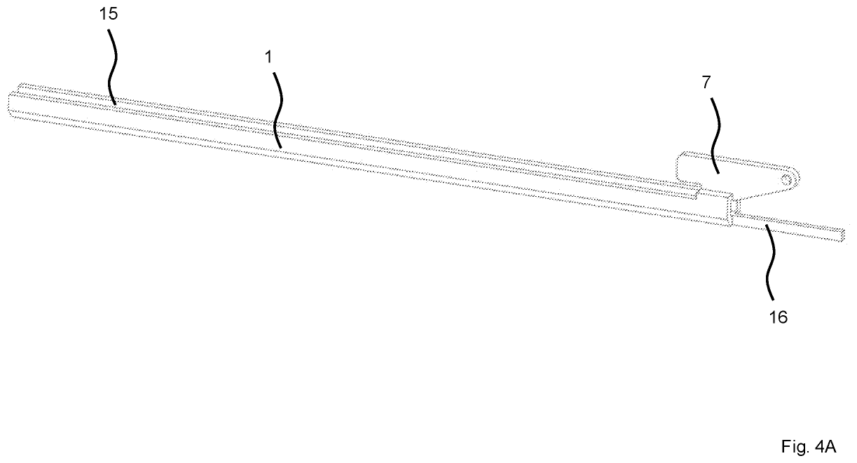

FIG. 4A shows a schematic illustration of a proximal arm segment of an articulating robotic arm;

FIG. 4B shows a schematic detail view of the proximal arm segment of FIG. 4A;

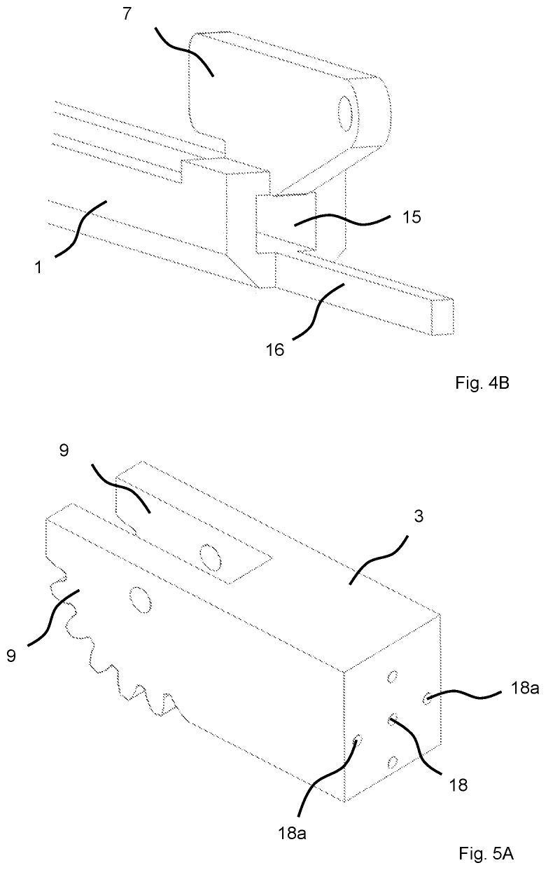

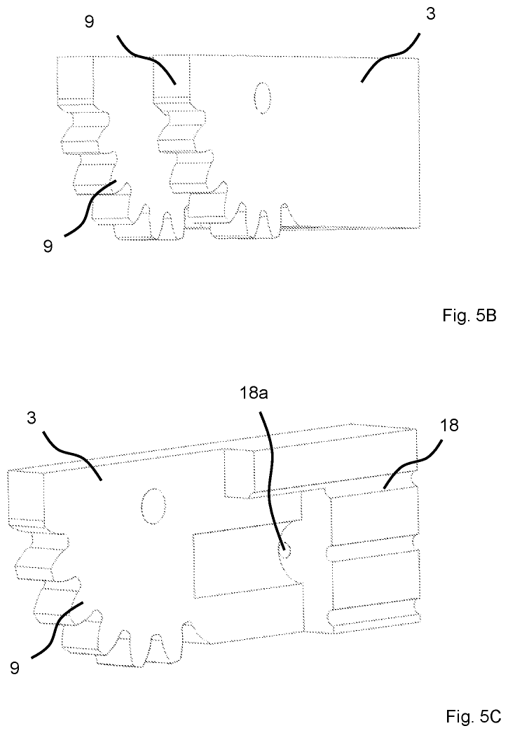

FIG. 5A shows a schematic illustration of an element of a distal arm segment of an articulating robotic arm;

FIG. 5B shows a schematic illustration of the element of FIG. 5A from a different perspective;

FIG. 5C shows a schematic sectional view of the element of FIG. 5A;

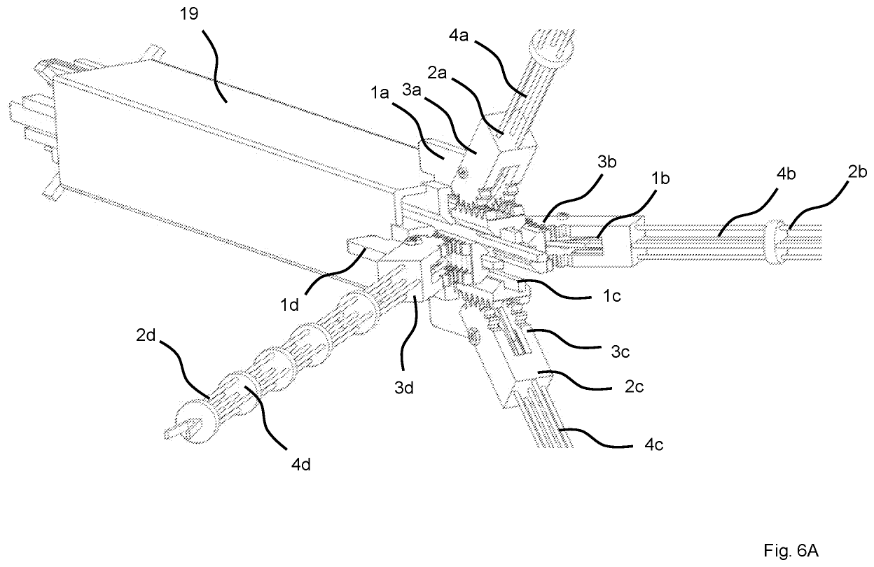

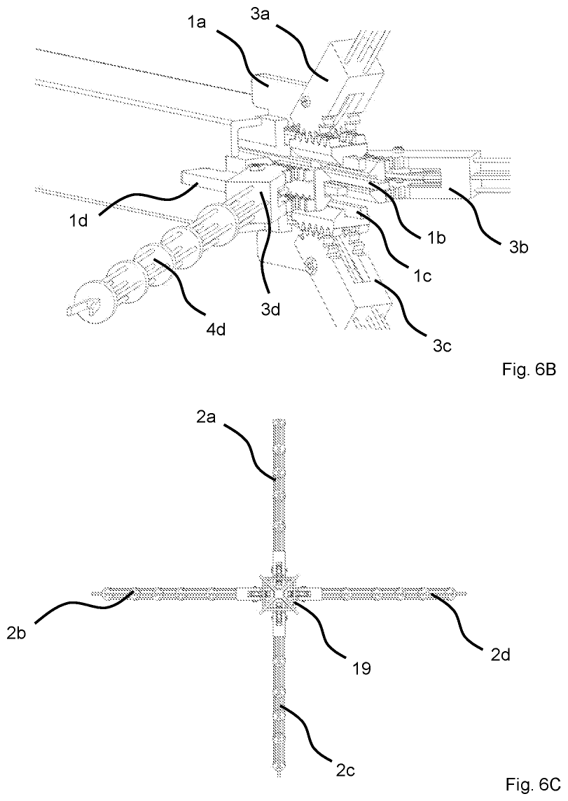

FIG. 6A shows a schematic illustration of a part of a surgical robot;

FIG. 6B shows a schematic detail view of the surgical robot of FIG. 6A;

FIG. 6C shows another schematic detail view of the surgical robot of FIG. 6A;

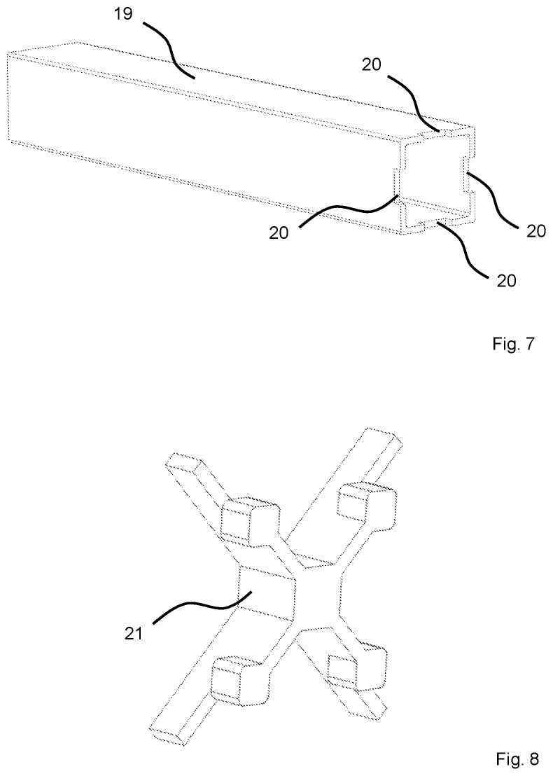

FIG. 7 shows a schematic illustration of a housing of a surgical robot;

FIG. 8 shows a schematic illustration of a fixing element for a surgical robot;

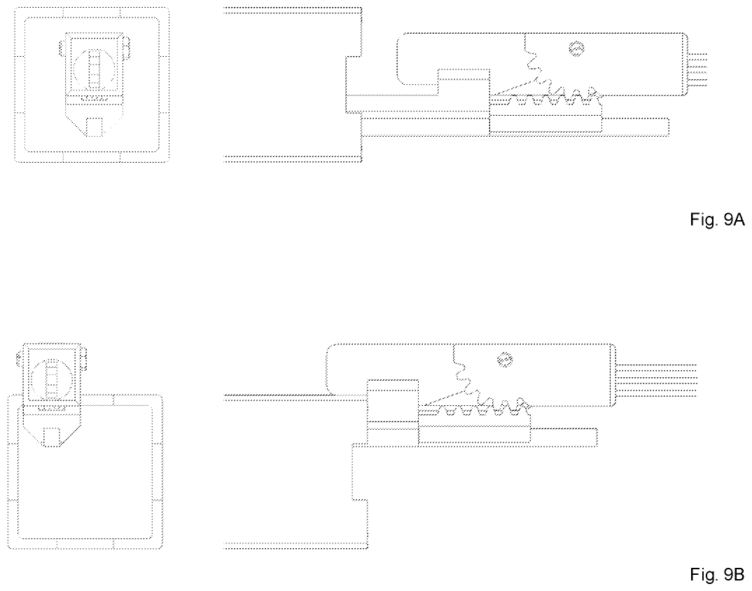

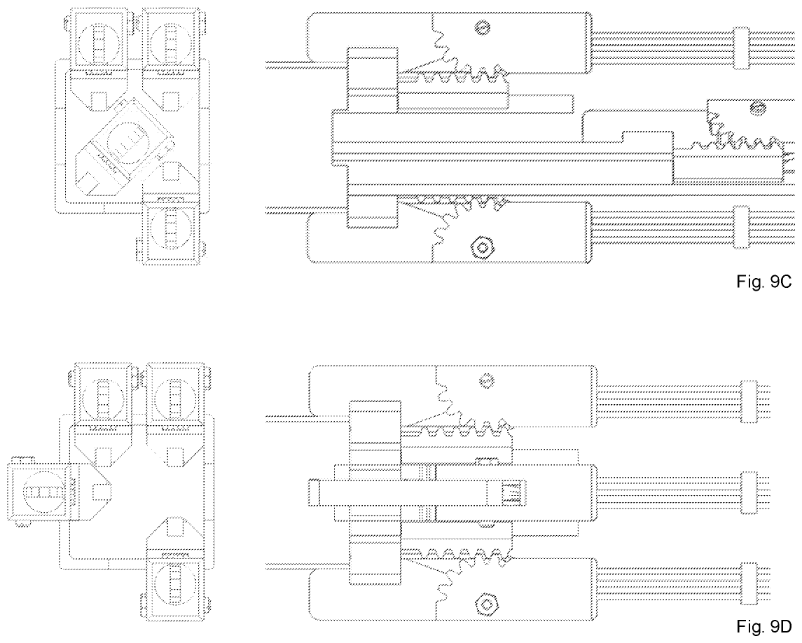

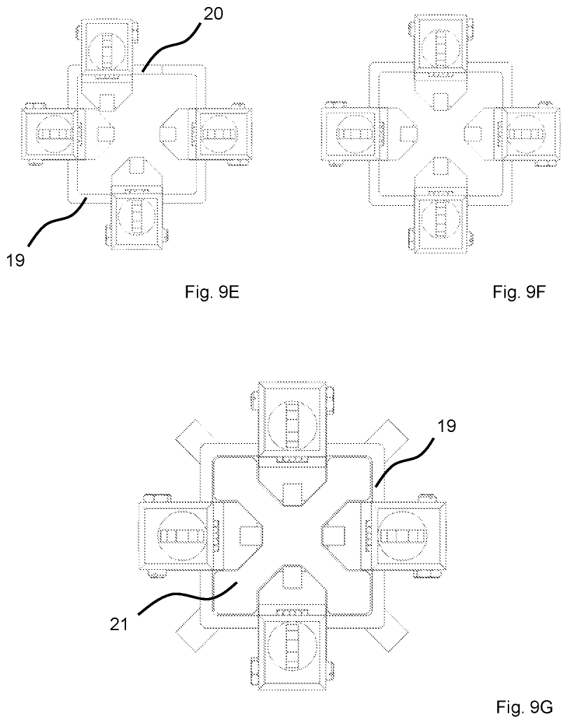

FIGS. 9A to 9G show schematic illustrations of the assembly procedure of a surgical robot for assembly in the body of a patient;

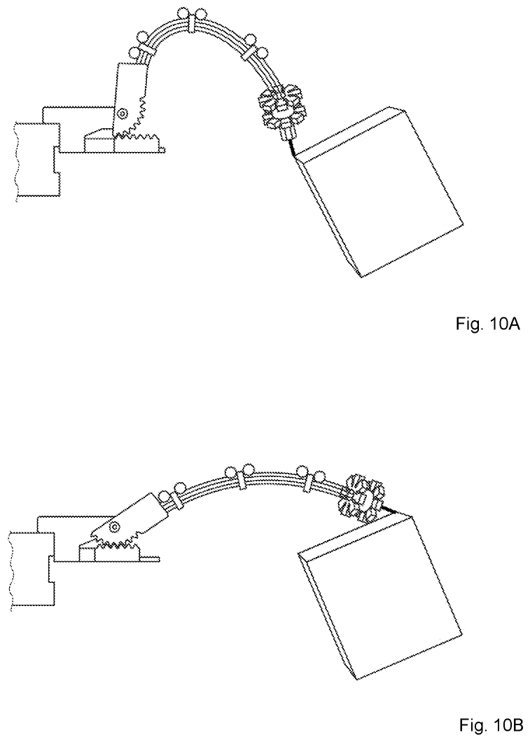

FIG. 10A shows a schematic view of an articulating robotic arm with a large angular deflection of a distal arm segment and a large bend of a flexible arm segment of the distal arm segment; and

FIG. 10B shows a schematic view of the angular robotic arm of FIG. 10A with a small angular deflection of the distal arm segment and a small bend of the flexible arm segment of the distal arm segment.

FIG. 1 shows an embodiment of an articulating robotic arm for minimally invasive surgery. The robotic arm is formed with a proximal arm segment 1 and a distal arm segment 2. Here, the proximal arm segment 1 is formed as a rigid segment. The distal arm segment 2 is formed with a fixed segment 3 and a flexible arm segment 4 connected distally thereto. The flexible arm segment 4 has five NiTi wires serving as pull-push means or elements 5. Two pairs of push-pull elements 5 each of which act antagonistically, are arranged around a central axis of the flexible arm segment 4. Pulling on one of the pull-push means 5 of a pair while simultaneously pushing on the other pull-push elements 5 of the pair causes an angular deflection in the form of a bend of the flexible arm segment 4. By means of pushing and pulling on a pull-push means 5 running on the central axis of the flexible arm segment 4, an end effector 6 of the robotic arm is actuated, which in the exemplary embodiment of FIG. 1 is a forceps element whose forceps jaws are opened and closed to provide a gripping movement.

As can be seen in the detail view of FIG. 2A, the proximal arm segment 1 and the distal arm segment 2 are connected to each other in an angularly deflectable manner. For this purpose, two flanks of the fixed segment 3 are arranged around a connection protrusion 7 and are pivotably connected to the connection protrusion 7 by means of a screw connection 8 passing through the flanks and the connection protrusion 7. The screw connection 8 thus defines a pivot axis of the distal arm segment 2 with respect to the proximal arm segment 1. For angularly deflecting the distal arm segment 2 with respect to the proximal arm segment 1, the distal arm segment 2 has two pinion segments 9 formed from the flanks, which are formed with pinion teeth 10, wherein the pinion teeth 10 are arranged around the pivot axis corresponding to a pinion. Rack teeth 11 of a rack element 12 engage with the pinion teeth. As a result of this, an angular deflection of the distal arm segment 2, namely the fixed segment 3, relative to the proximal arm segment 1 about the pivot axis can be achieved by means of a linear movement of the rack element 12, wherein the linear or translational movement of the rack teeth 11, via the engagement with the pinion teeth 10, cause a rotation of the pinion segments 9 and thus of the fixed segment 3 about the pivot axis. This angular deflection of the distal arm segment 2 is in principle independent of any angular deflection caused by a bending of the flexible arm segment 4, wherein, however, a relative change in the path of the pull-push means 5 when the fixed segment 3 is angularly deflected can result in an influence on the flexible arm segment 4.

The detail views of FIGS. 2B and 2C illustrate the angular deflection of the distal arm segment 2 with respect to the proximal arm segment 1 and the linear guidance of the rack element 12 during the angular deflection. In this connection, FIG. 2B illustrates a view of the proximal end of the proximal arm segment 1. FIG. 2C illustrates a view of the distal end of the proximal arm segment 1 with the distal arm segment 2 pivotably disposed thereon. It can be seen here that the pull-push elements 5 of the flexible arm segment 4 are guided along the entire length of the proximal arm segment 1 up to the proximal end thereof. Here, linear actuators can be operatively connected to the pull-push elements 5 to move them linearly and thus to actuate the flexible arm segment 4.

As shown in FIG. 3, the rack element 12 is formed with a narrow proximal rack segment 13 and a wider distal rack segment 14. Here, two sets of rack teeth 11 are formed on opposite sides of the distal rack segment 14, each of which engages pinion teeth 10 of one of the pinion segments 9 of the distal arm segment 2 in the articulating robotic arm.

FIG. 4A shows the proximal arm segment 1. FIG. 4B is a detail view of the distal end of the proximal arm segment 1. A guide channel 15, which receives the narrow proximal rack segment 13 for linearly guiding the rack element 12 in the longitudinal direction of the proximal arm segment 1, is formed in the proximal arm segment 1. The guide channel 15 is formed with a cross-section approximating a U-shape and the cross-section of the proximal rack segment 13 is adapted to the cross-section of the guide channel 15, as can be seen in FIG. 2B, for example.

Additionally, the proximal arm segment 1 has a guide rod 16 which is linearly guided in a rack element guide channel 17, thereby providing additional guidance of the translational movement of the rack element 12 for the angular deflection of the distal arm segment 2. The guide rod 16 is arranged at the distal end of the proximal arm segment 1 and protrudes therefrom in the longitudinal direction. The rack element guide channel 17 is formed on the underside of the wide distal rack segment 14, and the cross-sectional shapes of the guide rod 16 and the rack element guide channel 17 are matched with one another such that the rack element 12 with the rack element guide channel 17 can run on the guide rod 16 in the sense of a linear guide, as shown in FIGS. 2A and 2C, for example. In the exemplary embodiment shown, the rack element 12 and the rack element guide channel 17 have rectangular cross-sections. Alternatively, other cross-sectional shapes are also suitable, such as a U-shape or a V-shape.

FIGS. 5A and 5B show a fixed segment 3 of a distal arm segment 2 from different perspectives. On a side of the fixed segment opposite in the longitudinal direction to the pinion segments 9 formed on the flanks, guide holes 18 are formed which receive and linearly guide the pull-push elements 5 of the flexible arm segment 4. The pull-push elements 5 are guided through the guide holes from the flexible arm segment 4 to the proximal arm segment 1, and through the latter to the proximal end thereof, as can be seen in FIGS. 2A, 2B and 2C. At the proximal end of the proximal arm segment 1, the pull-push elements 5 can then be connected to linear actuators to drive and control the flexible arm segment 4.

FIG. 5C shows a sectional view of the fixed segment 3, in which it can be seen that the guide holes 18 are through-holes. Here, two guide holes 18a of the embodiment shown run partially through the flanks with the pinion segments 9 and open into a recess in the flanks, respectively. In FIG. 5C, it can be seen that the respective push-pull elements 5 can be guided in these guide holes 18a through a distal wall of the fixed segment 3 and then guided away from the respective flank by a slight bend.

When the fixed segment 3 is angularly deflected with respect to the proximal arm segment 1, a path from the proximal end of the proximal arm segment 1 to the distal end of the fixed segment 3 can change slightly for some of the pull-push elements 5, depending on their guidance along the robotic arm, which in turn can lead to a slight actuation of the flexible arm segment 4. Such a slight actuation of the flexible arm segment 4 can either be taken into account and used in an overall movement of the robotic arm, or it can be provided to compensate for the path change by a corresponding actuated countermovement of the respective pull-push element, thus avoiding an unwanted actuation of the flexible arm segment 4.

FIG. 6A shows a surgical robot with four articulating robotic arms according to the disclosure. Here, the four proximal arm segments 1a, 1b, 1c, 1d of the robotic arms are arranged over the major part of their respective longitudinal extent in an enveloping housing 19. The distal arm segments 2a, 2b, 2c, 2d with the fixed segments 3a, 3b, 3c, 3d and the flexible arm segments 4a, 4b, 4c, 4d are arranged outside the housing 19 and can move during an operation with the surgical robot.

In FIG. 6B, an angular deflection of the respective fixed segments 3a, 3b, 3c, 3d with respect to the respective proximal arm segments 1a, 1b, 1c, 1d is shown in detail. By means of an angular deflection of the respective fixed segments 3a, 3b, 3c, 3d, the flexible arm segments 2a, 2b, 2c, 2d can first be moved apart and subsequently, by bending the flexible arm segments 2a, 2b, 2c, 2d of their end effectors, can be brought back to each other at the surgical site from different directions, whereby surgical movements in the course of so-called triangulation can be facilitated compared to parallel guidance of the robotic arms to the surgical site.

FIG. 6C shows the surgical robot from a perspective from a proximal end. The proximal end of the surgical robot can be connected to other components for actuation of the robotic arms, in particular to a drive unit with actuators for driving the various degrees of freedom of the robotic arms.

In the embodiment shown, the surgical robot is a robot that can be assembled or mounted in the body of a patient. In this case, the articulating robotic arms are inserted individually into the patient's body and only thereafter are connected to one another in the housing 19 to form the surgical robot.

FIG. 7 is a detail view of the housing 19 of the surgical robot. It can be seen here that the housing 19 has recesses 20 at a distal end, which serve to mount the surgical robot, as explained in detail below.

FIG. 8 shows a sealing fixing element 21 which, after arranging a plurality of robotic arms in the housing 19, is inserted into a proximal end of the housing 19 to correctly position the robotic arms in relation to each other in the housing and to fix them in this position. At the same time, the fixing element 21 acts in a sealing manner. In particular, this can prevent insufflation gas from escaping from the patient's abdominal cavity between the robotic arms through the housing 19 during use, i.e., during an operation. For example, the fixing element 21 can be provided with a rubber coating for sealing purposes.

FIGS. 9A to 9G illustrate a mounting or assembly process of the surgical robot. Here, the robotic arms are inserted one after the other into the distal end of the housing 19. As can be seen in FIGS. 9A and 9B, the first robotic arm is inserted centrally into the housing 19 and then moved to the edge of the housing 19. In this manner, the other robotic arms can also be inserted into the housing 19 and moved to the edge of the housing 18. Insertion of the fourth robotic arm requires a twist to insert it through the cavity between the three robotic arms al-ready inserted and to subsequently move it to the edge of the housing 19 This can be seen in detail in FIGS. 9C and 9D.

Thereafter, a rearrangement of the robotic arms in the housing 19 takes place, as shown in FIGS. 9E and 9F, so that each of the robotic arms is arranged in an assigned mounted position. When arranging the robotic arms at the edge of the housing 19, as can be seen in FIGS. 9A to 9D, the respective connecting protrusion 7 of the respective robotic arm is pushed over the edge of the housing 19, thereby connecting the robotic arm to the edge of the housing. After rearrangement of the robotic arms, the respective connecting protrusions 7 are slid into the assigned recesses 20, thereby fixing the robotic arms in their mounted position at the distal end of the housing 19.

In alternative configurations, the robotic arms can be miniaturized such that they can be inserted directly into the housing 19 in their final arrangement. In this case, the step of rearranging during mounting can be omitted.

Finally, the sealing fixing element 21 is arranged in the proximal end of the housing 19 as shown in FIG. 9G, wherein FIG. 9G is a view of the distal end of the housing 19 so that the fixing element 21 is shown partially covered. The fixation element both fixes the robotic arms in their mounted arrangement to the proximal end of the housing 19 and maintains insufflation during an operation by means of the seal.

In alternative configurations, a surgical robot can be provided that is not mountable within a patient's body. For example, robotic arms can be arranged on arms of robots known as such, which remain completely outside the patient also during surgery and provide additional degrees of freedom.

In FIGS. 10A and 10B, an embodiment of an articulating robotic arm for minimally invasive surgery is shown in different states of movement. According to FIG. 10A, the fixed segment 3 is angled at an approximately right angle with respect to the proximal arm segment 1. It can be seen here that the rack element 12 is pushed far forward. In addition, the flexible arm segment 4 is strongly bent. The distal end of the robotic arm holds a weight. Compared to the state shown in FIG. 10A, the fixed segment 3 according to FIG. 10B is angled by a much smaller angle with respect to the proximal segment, and the flexible arm segment 4 is less strongly bent.

Load capacity tests were carried out with an exemplary embodiment of an articulating robotic arm. In this case, the structure of the robotic arm and the forms of movement corresponded to the illustrations in FIGS. 10A and 10B, with the proximal arm segment 1, the fixed segment 3 and the rack element 12 being produced by means of 3D printing. The robotic arm consists of proximal arm segment, distal arm segment with fixed segment, a screw connection, a rack, and a flexible arm segment. A pinion is integrated into the distal arm segment. The teeth of the rack and the teeth of the pinion engage with each other. The rack is guided by means of a guide channel. The flexible arm segment consists of discs. The discs have two functions: They ensure radial spacing between individual NiTi wires, and bending can be achieved by attaching all wires to the end of the flexible arm segment. An end disc is attached to both sides of each NiTi wire.

In the tests, the angle adjustment unit showed high functionality. The NiTi wires proved to be the weakest point. The meshing between the rack and pinion was flawless at all times. Although the angle adjustment mechanism was subjected to a double load due to the bending of the NiTi wires as well as the lifting of a load with a mass of 30 g and with the teeth being relatively flat with a height of approx. 1.5 mm, power was transmitted at all times. The minimal play between the pinion teeth and the rack teeth was sufficient to ensure smooth movement. Due to the guidance by guide rail, position-independent stability of the rack was achieved.

When the weight was attached directly to the end of the distal arm segment, i.e., to a base plate, loads of 200 g could be lifted without any problems. From this, it can be concluded that the angular deflection mechanism is suitable for use in minimally invasive surgery. Herein, it was unexpected that the combination of rack, pinion, and guide rail exhibited the established functionality as an angular adjustment mechanism, even with a high load of 200 g. The gearing proved to be reliable and positioning commands were implemented.

The features disclosed in the foregoing description, the claims, and the drawing can be relevant in the implementation of the various embodiments, either individually or in any combination.

1. An articulating robotic arm for minimally invasive surgery, with
a proximal arm segment extending along a longitudinal axis of the robot arm and
a distal arm segment connected to the proximal arm segment, pivotable about a pivot axis which is orthogonal to the longitudinal axis,
wherein
the distal arm segment comprises a pinion segment formed with pinion teeth arranged about the pivot axis in the form of a pinion; and
a rack element is arranged on the proximal arm segment in such a manner that
the rack element is movable in the longitudinal direction of the rack element parallel to the longitudinal axis by means of a translation, and
rack teeth formed on the rack element are engaged with the pinion teeth so that a movement of the rack element in the longitudinal direction via the rack teeth and the pinion teeth is converted into a rotational movement of the distal arm segment about the pivot axis and so that the distal arm segment pivots relative to the proximal arm segment about the pivot axis.

2. The articulating robotic arm according to claim 1, wherein the pinion teeth are formed as protrusions of the distal arm segment.

3. The articulating robotic arm according to claim 1, wherein the distal arm segment has a second pinion segment with pinion teeth arranged around the pivot axis, which second pinion segment is arranged spaced apart along the pivot axis from the pinion segment on the distal arm segment.

4. The articulating robotic arm according to claim 3, wherein the rack element has two sets of rack teeth, wherein the two sets of rack teeth are arranged parallel to and spaced apart from each other in the direction of the pivot axis such that the first set of rack teeth engages with the pinion teeth of the pinion segment and the second set of rack teeth engages with the pinion teeth of the second pinion segment.

5. The articulating robotic arm according to claim 1, wherein the rack element is formed with a proximal rack segment and a distal rack segment connecting to the proximal rack segment in the longitudinal direction, wherein the distal rack segment is wider in the direction of the pivot axis than the proximal rack segment.

6. The articulating robotic arm according to claim 1, wherein the proximal arm segment has a guide channel extending along the longitudinal axis in which the rack element, in particular with a proximal rack segment, is arranged and guided at least in sections.

7. The articulating robotic arm according to claim 1, wherein
the proximal arm segment has an arm segment guide element, and
the rack element has a rack element guide channel,
wherein the arm segment guide element is arranged and guided at least in sections in the rack element guide channel.

8. The articulating robotic arm according to claim 1, wherein the distal arm segment is formed at least in sections as a flexible arm segment which can be angularly deflected continuously or quasi-continuously along its longitudinal extent.

9. The articulating robotic arm according to 8, wherein the flexible arm segment is formed with at least three pull-push elements which are arranged parallel along a longitudinal extent of the flexible arm segment and movable with respect to each other along the longitudinal extent.

10. The articulating robotic arm according to claim 1, wherein the distal arm segment is formed at least in sections as a discrete-robotic arm segment with segments arranged one behind the other along its longitudinal extent, wherein the discrete-robotic arm segment can be angularly deflected in segments along its longitudinal extent.

11. The articulating robotic arm according to claim 1, comprising a linear actuator connected to the rack element and configured to translationally move the rack element back and forth parallel to the longitudinal axis.

12. A surgical robot, in particular for minimally invasive surgery, with an articulating robotic arm according to claim 1.

13. The surgical robot according to claim 12, wherein the surgical robot is designed as a robot for assembly inside the body of a patient.

14. A method for producing an articulating robotic arm, comprising the steps of:
providing a proximal arm segment whose direction of extent defines a longitudinal axis of the robotic arm;
providing a distal arm segment comprising a pinion segment formed with pinion teeth arranged about a pivot axis in the form of a pinion;
connecting the distal arm segment to the proximal arm segment such that the distal arm segment is pivotable about the pivot axis relative to the proximal arm segment and the pivot axis extends orthogonal to the longitudinal axis; and
arranging a rack element on the proximal arm segment such that
the rack element is movable in the longitudinal direction of the rack element parallel to the longitudinal axis by means of a translation, and
rack teeth formed on the rack element are engaged with the pinion teeth so that a movement of the rack element in the longitudinal direction via the rack teeth and the pinion teeth is converted into a rotational movement of the distal arm segment about the pivot axis and the distal arm segment pivots relative to the proximal arm segment about the pivot axis.
